(19) United States
(12) Patent Application Publication
(10) Pub. No.: US 2005/0182352 A1
(43) Pub. Date: Aug. 18, 2005

(54) Dialysis catheter tip

(76) Inventors: Kristian DiMatteo, Waltham, MA (US); Todd Beaupre, Reading, MA (US); James Culhane, Westborough, MA (US); Benjamin Bell, Haverhill, MA (US); James Weldon, Roslindale, MA (US)

Correspondence Address: Patrick Fay, Esq.;FAY KAPLUN & MARCIN, LLP, Suite 702, 150 Broadway, New York, NY, 10038, US

(21) Appl. No.: 10/777,545

(22) Filed: Feb. 12, 2004

(51) Int. Cl. A61M 3/00

(52) U.S. Cl. 604/43

(57) ABSTRACT

A distal tip for a catheter comprises first and second lumens extending therethrough, wherein in an operative configuration, the first and second lumens are coupled to first and second lumens of a dual lumen catheter with a first opening of the distal tip fluidly connected to the first lumen for inflow of fluid from a body lumen into which the distal tip is inserted in a normal mode of operation and for outflow of fluid thereto in a reverse mode of operation and a second opening fluidly connected to the second lumen. The second opening is disposed distally from the first opening and separated therefrom by a selected stagger distance for outflow of fluid therefrom when the catheter is in the normal mode of operation and for inflow of fluid from the body lumen in a reverse mode of operation. The distal tip also includes a contoured flow deflection element directing, in the reverse mode of operation, outflow from the first opening away from the second opening and a contoured outlet portion of the second opening reducing an outflow velocity therefrom in the normal mode of operation.

### BACKGROUND OF THE INVENTION

Medical procedures for the treatment of chronic diseases often require repeated access to the vascular system for the injection of therapeutic compounds and the sampling of blood. Kidney dialysis, chemotherapy and other chronic treatments generally rely on catheters for both injection to and withdrawal of fluids from the vascular system. For example, during a kidney dialysis treatment, large amounts of blood are withdrawn from the patient and treated externally in a dialysis machine to remove impurities and add nutrients, medications and any other desired therapeutic elements. This treated blood is then returned to the patient.

Typically, a single catheter having two or more lumens is used for the removal and return of the blood with a first of the lumens being used to aspire impure blood from a blood vessel (usually a vein) and a second of the lumens being used to return the treated blood to the blood vessel. A single needle including inlet and outlet orifices connected to the first and second lumens, respectively, is commonly used to perform both functions simultaneously.

Since the inlet and outlet orifices are located on the same needle, a certain amount of recirculation may occur. That is, a portion of the treated blood exiting the outlet orifice is returned directly to the inlet orifice to return to the dialysis machine. This delays treatment of portions of the venous blood which is displaced by the recirculating fluid thereby increasing the time required to achieve a desired amount of purification and, consequently, increasing the cost of the procedure and patient discomfort.

### SUMMARY OF THE INVENTION

In one aspect, the present invention is directed to a distal tip for a catheter comprising first and second lumens extending therethrough, wherein in an operative configuration, the first and second lumens are coupled to first and second lumens of a dual lumen catheter and a first opening fluidly connected to the first lumen for inflow of fluid from a body lumen into which the distal tip is inserted in a normal mode of operation and for outflow of fluid thereto in a reverse mode of operation in combination with a second opening fluidly connected to the second lumen, the second opening being disposed distally from the first opening and separated therefrom by a selected stagger distance for outflow of fluid therefrom when the catheter is in the normal mode of operation and for inflow of fluid from the body lumen in a reverse mode of operation and a contoured flow deflection element directing, in the reverse mode of operation, outflow from the first opening away from the second opening. A contoured outlet portion of the second opening reduces an outflow velocity therefrom in the normal mode of operation.

### BRIEF DESCRIPTION OF THE DRAWINGS

FIG. 1 is a side elevation view of a dual lumen catheter according to an embodiment of the present invention;

FIG. 2 is a perspective view of the dual lumen catheter shown in FIG. 1;

FIG. 3 is a cross sectional view showing the elongated body of the catheter along line III-III;

FIG. 4 is a cross sectional view showing the catheter along line IV-IV;

FIG. 5 is a schematic diagram showing the fluid flow around the catheter according to an embodiment of the invention in a normal mode;

FIG. 6 is a schematic diagram showing the fluid flow around the catheter of FIG. 5 in a reverse mode;

FIG. 7 is a cross sectional side elevation view of another embodiment of a catheter tip according to the present invention;

FIG. 8 is a cross sectional side elevation view of an intermediary step in the construction of a catheter tip according to a different embodiment of the invention;

FIG. 9 shows a top plan view of the distal portion of the intermediary step shown in FIG. 8;

FIG. 10 shows a front elevation view of the distal portion of the intermediary step shown in FIG. 9;

FIG. 11 shows a cross sectional side elevation view of a different embodiment of the catheter tip according to the invention;

FIG. 12 shows a side elevation view of an alternative exemplary manufacturing method for a catheter tip according to the invention; and

FIG. 13 shows a side elevation view of another alternative manufacturing method for a catheter tip according to the invention.

### DETAILED DESCRIPTION

The present invention may be further understood with reference to the following description and the appended drawings, wherein like elements are referred to with the same reference numerals. The present invention is related to medical devices that are used to access the vascular system of a patient. Although the present invention is described in regard to a catheter used to withdraw and return blood to the patient during dialysis, those skilled in the art will understand that the invention is equally applicable to any treatment in which a single catheter is used for the withdrawal of fluid from and the provision of fluid to a blood vessel or other body lumen. More particularly, the invention is related to catheter tips that minimize the amount of recirculation taking place during such treatments.

To reduce recirculation, the tips of conventional dialysis catheters are shaped, to a certain extent, to separate the inlet and outlet orifices. For example, conventional designs have used a staggered arrangement of the orifices, with the outlet orifice further downstream (in the direction of the flow of blood) than the inlet orifice. Typically, this configuration results in the outlet orifice being placed on the needle distally of the inlet orifice. However, at times it is necessary to reverse the direction of flow through the catheter so that the inlet orifice serves as an outlet and the outlet orifice serves as an inlet.

In this reverse mode, the outlet orifice is no longer downstream from the inlet aspiring non-treated blood, increasing the amount of recirculation. This effect is alleviated to a certain extent by the flow of blood which tends to entrain the injected blood away from the needle. However, the flow of blood pulsates with the beating heart and, when the rate of flow is at its lowest, the purified blood exiting the conventional catheter is not entrained away from the needle and the inlet through which it may be recirculated.

To gain a quantitative understanding of the scope of the problem caused by recirculating blood, exemplary recirculation rates determined experimentally are describe below. For an exemplary conventional staggered tip catheter with inlet and outlet orifices displaced longitudinally relative to one another, the recirculation rate in the normal more of operation is about 0.4% while for the reverse mode of operation the recirculation rate is about 20.9%. In contrast, exemplary embodiments of a catheter tip according to the present invention provide recirculation rates in the normal mode of between about 0.4% and 2.4%, with reverse mode recirculation rates of between about 6.3% and about 7.8%. As can be seen, the exemplary embodiments according to the present invention provide a substantial reduction of the amount of blood (or other fluid or mixture of fluids) which recirculates in the reverse mode of operation of the catheter, while maintaining a normal mode recirculation comparable to that of the conventional catheters.

In addition to the amount of recirculation in both reverse and normal modes of operation, thrombogenicity of the design is of interest. This refers to the tendency of the catheter tip to facilitate coagulation of the blood flowing therethrough, and form coagulated particles known as thrombi. As is understood by those skilled in the art, thrombi may be very dangerous for the patient as they can become dislodged and travel through the body. The hemolysis of the catheter tip (i.e., the tendency of the tip to damage blood cells flowing therethrough) is also important.

The exemplary embodiments of the present invention thus provide improvements in the ability of the catheter to minimize recirculation in a reverse mode of operation, while at the same time retaining the ability to minimize recirculation in the normal mode of operation. Those skilled in the art will understand that this latter property is important as the catheter spends a majority of its operational life in the normal mode of operation with the reverse mode of operation being implemented less frequently. In addition, the embodiments of the catheter tip according to the present invention retain acceptable thrombogenicity and hemolysis properties.

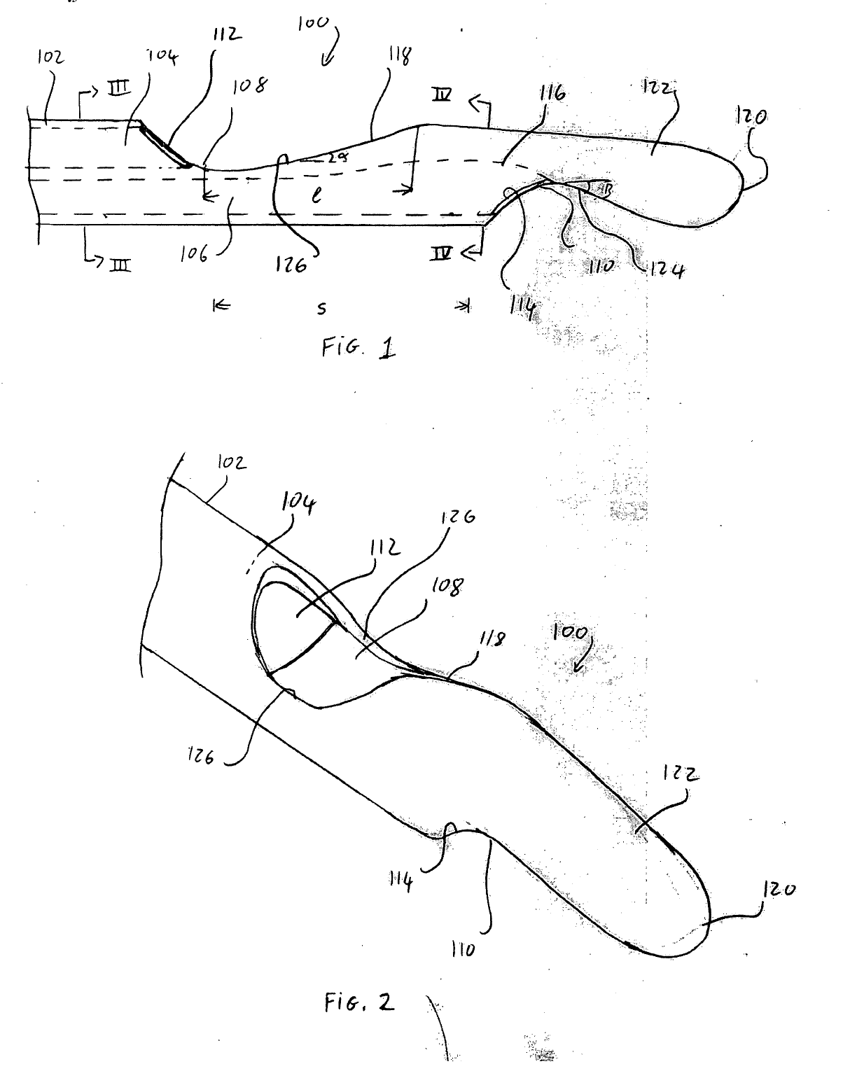

FIGS. 1 and 2 depict a tip 100 for a dialysis catheter (not shown) comprising a proximal substantially tubular portion 102 designed to provide a transition to the elongated tubular body of the catheter as will be described below. The tip 100 reduces recirculation of blood in the reverse mode of operation through a novel shaping of first and second openings 108, 110 which, in the normal mode of operation, act respectively as inlet and outlet openings of the catheter. Additional control over recirculation is gained by providing in the tip 100 a flow control element 122 shaped to achieve one or more of several goals. For example, the flow control element 122 may be designed to deflect a flow from the first opening 108 away from the tip 100, and particularly away from the second opening 110. In the reverse mode of operation this feature prevents flow exiting the first opening 108 from being ingested by the second opening 110. The flow control element 122 may also be designed to reduce recirculation in the normal mode of operation by deflecting fluid exiting the second opening 110 away from the first opening 108.

In addition to FIGS. 1-4, the normal and reverse modes of operation of the tip 100 are depicted in FIGS. 5 and 6. FIG. 5 shows the normal mode of operation where a second lumen 106 of the tip 100 which is connected fluidly with the second (outlet) opening 110, ejects the fluid into a bloodstream traveling in the direction shown by the arrow B. Aspiration of the untreated blood occurs through the first (inlet) opening 108 which is connected to a first lumen 104 of the tip 100. FIG. 6 shows the reverse mode of operation, where the first lumen 104 and the first opening 108 are used to inject blood into a patient's vein, while the second lumen 106 and the second opening 110 are used to aspire blood therefrom. As will be described in greater detail below, the location and shape of the first and second openings 108, 110 as well as the shape of the flow control element 122 cooperate to obtain desired characteristics of the catheter tip 100.

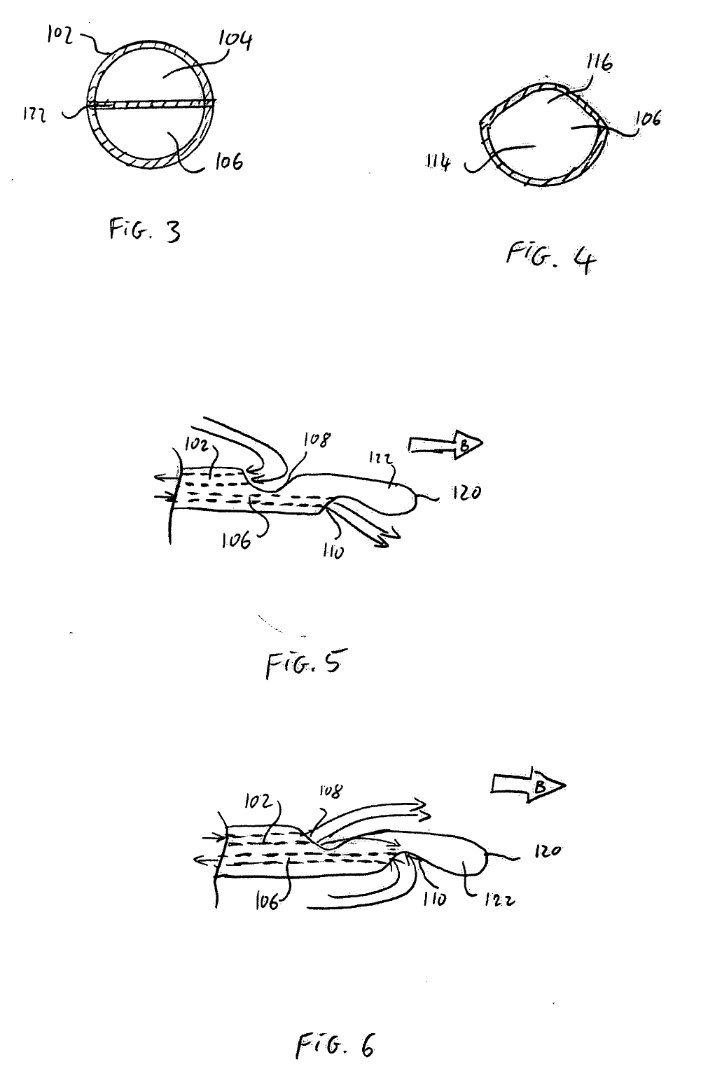

FIG. 3 shows a cross sectional area along line III-III of the proximal portion 102 of the catheter tip 100 near a location where the tip 100 transitions to the elongated body of the catheter. The first and second lumens 104 and 106 are shown in an exemplary configuration, each having a substantially ‘D’ shaped cross sectional area. This configuration is compatible with a conventional catheter having a circular cross section and two lumens of approximately equal dimensions. It will be apparent to those skilled in the art that different cross sectional shapes may be used in the proximal portion 102 of the tip 100 depending on the shape of the catheter used and the shapes of the lumens therein. Different methods of connecting or integrating the tip 100 into the catheter may also be used, as will be described below.

In greater detail, the flow control element 122 may include a ramp 118 located in proximity to the first opening 108, as shown in FIG. 1. The ramp 118 is preferably oriented so that in the reverse flow mode, fluid exiting from the first opening 108 is deflected upward, away from the main body of the tip 100. Those skilled in the art will understand that, in this context, the directions “up” and “down” are used simply in relation to the orientation of the drawings and do not refer to the orientation of any features when in use. The actual orientation of the components of the tip 100 may be similar, inverted, or shifted sideways relative to the orientation shown. The ramp 118 may have a length l selected to provide a desired deflection of the flow. Similarly, the ramp 118 may have a ramp angle α also selected to obtain the desired deflection. The angle α may be constant throughout the length of the ramp 118 or may be vary therealong. As would be understood by those skilled in the art, the specific shape, length l and angle α of the ramp 118 may be selected based on the specific application for which a catheter including the tip 100 is intended. For example, these characteristics may be varied based on the expected blood flow rate, inlet and outlet flow rate, and desired performance of the catheter in the normal and reverse modes of operation.

The flow control element 122 may also include lateral elements 126 designed to prevent the flow from “wrapping” around the sides of the tip 100 toward the second opening 110. The first opening 108 includes an orifice 112 formed on a plane diagonal to a longitudinal axis of the first lumen 104. The specific angle and size of the orifice 112 may be selected to cooperate with the ramp 118 and to obtain a selected flow rate out of the first opening 108. The length of the flow control element 122 in front of the ramp 118 may also be selected in part to reduce the tendency of the flow of blood to recirculate during the reverse mode of operation. In addition, a contoured bolus 120 may be provided at a distal-most point of the tip 100 to facilitate insertion of the tip/catheter assembly into the patient's vein, and to assist in navigating the assembly therein. Preferably, the contoured bolus 120 forms an atraumatic tip for catheter tip 100 allowing the catheter tip 100 to penetrate and navigate within the patient's blood vessels without causing injury to the blood vessel walls.

Another important consideration in the design of the catheter tip 100 is the stagger distance s between the first and second openings 108, 110. An increase in the stagger distance s generally results in a reduction in recirculation. However, if the stagger distance s is increased excessively, the resulting catheter tip 100 will become impractical for use in a patient's blood vessel (i.e., the length of the tip 100 will make navigation difficult or impossible). Accordingly, an optimum stagger distance s may be determined for various applications. For example, the stagger distance s may have a dimension of between about 1 cm to about 2 cm, for a dialysis catheter of typical dimensions while, for other applications to be carried out in vessels of greater or lesser diameter and with longer or shorter radii of curvature to be navigated, different optimum dimensions may be arrived at.

Additional control of the flow surrounding the tip 100 may be achieved by forming the flow control element 120 with a second ramp 124 designed to deflect flow exiting from the second opening 110 in the normal mode of operation of the catheter. The second ramp 124 or a similar flow control device may be used to further reduce the recirculation of blood in the normal mode by directing the exiting flow away from the first opening 108. For example, the second ramp 124 may have a length and a ramp angle β designed to cooperate with the orifice 114 of the second opening 110. For example the orifice 114 may be formed on a plane inclined with respect to a longitudinal axis of the second lumen 106 to form a substantial mirror image of the orifice 112 of the first opening 108. Properly forming the contours of the second ramp 124 further reduces the amount of recirculation existing in the normal mode. However, the design of the second opening 110 and the second ramp 124 may be less critical than the design of the first opening 108 and the first ramp 118 as, in the normal mode of operation, flow exiting the second opening 110 is entrained away from the first opening 108 by the natural flow of blood and is less likely to be aspired again.

The flow control element 122 may also be designed to include features adapted to increase an exit plane cross sectional area of the second opening 110. For example, an upper expanded section 116 may be included in the design, as shown in FIGS. 1 and 4. The upper expanded section 116 may be used to form a bulge or expansion of the second lumen 106, in a region near the orifice 114. The purpose of the upper expanded section 116 is to increase the cross sectional area at the exit of the second lumen 106 to reduce the velocity of the blood flow exiting the second opening 110 in the normal mode of operation. A lower outflow velocity is preferable because excessive flow velocity may damage the tissue upon which the flow impinges. Accordingly, providing an upper expanded section 116 or a similar structure allows for a high flow rate exiting the dialysis catheter, while reducing the possibility of tissue damage due to the high velocity outflow.

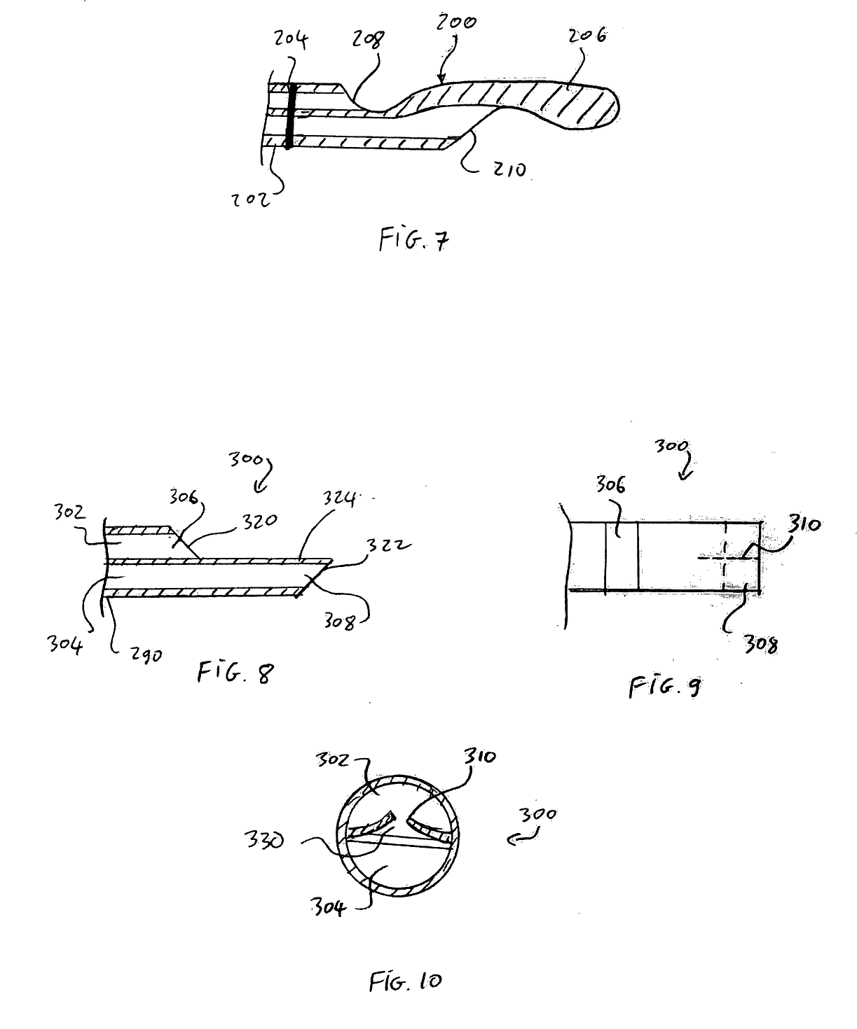

The manufacture and assembly of a catheter tip according to the present invention may be carried out in different ways. In one exemplary embodiment of the manufacturing process according to the present invention, a tip section 200 is manufactured separately from the rest of a dual lumen dialysis catheter 202. As shown in FIG. 7, the tip section 200 is completed and then attached to the catheter 202 with a tip to shaft joint 204. For example, the tip section 200 may be formed by molding and may be complete including a flow control element 206, a first opening 208 and a second opening 210. All or some of the features described above with respect to the tip embodiments shown in FIGS. 1-6 may be included in the complete tip section 200. In one exemplary embodiment, the tip section 200 may be formed of molded silicone. Alternatively, other polymers commonly used in manufacturing catheters may be used, such as, for example, carbothane.

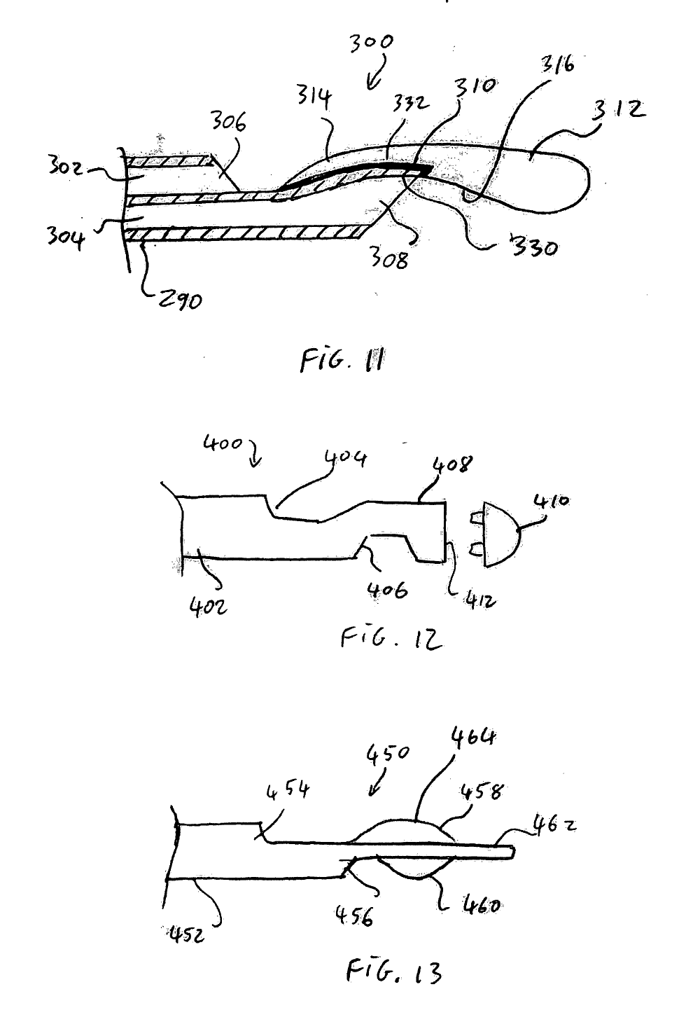

In some applications, particularly when carbothane is used as the material for manufacturing the catheter and the tip region, molding the entire tip structure and connecting it to a catheter as finishing step may not give satisfactory results. Accordingly, in a different embodiment of the invention, the tip structure may be formed in multiple steps. For example, in one embodiment the catheter shaft is retained all the way to the end of the distal tip, and is shaped to form the core of the tip portion. An overmolding process may be used to form the contoured bolus defining the flow control elements of the tip, according to the invention. As shown in FIGS. 8-11, a catheter tip 300 may be formed by modifying the distal end of a catheter 290, and then attaching only a small, separately formed, portion of the tip. In this exemplary assembly method, it is not necessary to mold the tip as a separate unit which is later attached to the catheter 290.

FIG. 8 shows the tip 300 of the catheter 290 in an initial step of fabrication. The distal portion of the catheter 290 is trimmed, for example, skived, to obtain a staggered configuration of the openings. In the exemplary embodiment, the first lumen 302 is cut along a plane 320, at a selected angle with a portion of the first lumen 302 distal from the plane 320 being removed such that a top surface 324 of the second lumen 304 is exposed. The second lumen 304 is cut along a plane 322 which may be, for example, at an angular orientation opposite to that of the plane 320. In this manner the first orifice 306 and the second orifice 308 are formed so that they point towards opposite sides of the tip 300. Alternatively, other manufacturing methods suitable to obtain the first and second orifices 306, 308 in the staggered configuration shown may be used. For example, the catheter 290 may be shaped during manufacture to have a distal end with staggered lumens.

A slit or web cut 310 may be formed in a subsequent step, along the distal end of an upper surface 324. The slit 310 may have a length appropriate to allow upward expansion of the second lumen 308, to form an upper expanded section 330 in a subsequent forming step. As discussed above, the upper expanded section 330 promotes a lower velocity of the flow exiting the second orifice 308 in the normal mode, by providing a larger exit plane cross sectional area of the second lumen 304. By cutting the slit 310 in the upper surface 324, a molding core or other tool may be inserted in the distal portion of the second lumen 304 to expand upwardly the distal portion. The size of the slit 310 may be determine based, for example, on the material forming the catheter 290 and on the desired maximum exit velocity of the flow leaving the second lumen 304.

FIG. 11 shows a later step in the formation of the distal tip 300 of the catheter 290. Here, a contoured bolus 312 is formed by overmolding on top of the upper surface 324 of the second lumen 304. In the exemplary embodiment, the molding process attaches the contoured bolus 312 to the catheter 290, and also forms the upper expanded section 330 by opening up the slit 310. According to this exemplary embodiment of the invention, the contoured bolus 312 defines a first ramp 314 which is designed to control and direct the flow exiting the first orifice 306, in the reverse mode of operation. The contoured bolus 312 may also define a second ramp 316 adapted to deflect and control the flow exiting the orifice 308, in the normal mode of operation. All the features described above with reference to different embodiments of the distal tip may be included in the flow deflection element 332 defined by the contoured bolus 312. Accordingly, the present embodiment also achieves a significant reduction in fluid recirculation in both the normal and the reverse modes of operation.

A different embodiment according to the invention is shown in FIG. 12. Here, a distal tip portion 400 is assembled from multiple parts. A catheter 402 is provided with a first orifice 404 and a second orifice 406 by skiving or by another known manufacturing process. The same process may also form a flow deflection element 408 attached to the distal end of catheter 402. A tip 410 may be formed separately, by molding, grinding or another suitable process, and may then be attached to a distal surface 412 of the catheter 402. The exemplary method results in a distal tip 400 comprising flow deflection portions for both the orifices 404 and 406, as well as a tip portion 410 shaped to facilitate insertion and navigation in the patient's blood vessels.

FIG. 13 shows yet another exemplary embodiment of a manufacturing process used in forming an improved distal tip 450 of a catheter, such as a dialysis catheter. In this example, the catheter 452 is skived to obtain the staggered configuration of the first and second orifices 454 and 456 shown and an extension 462 of a portion of the catheter 452 is left after skiving to provide a base upon which the flow control portion of the tip 450 is formed. It will be apparent to those of skill in the art that other manufacturing methods in addition to skiving may be employed to obtain a distal end of the catheter 452 as shown in FIG. 13. One or more bulbs of material may be deposited over the extension portion 462, such as an upper bulb 458 and a lower bulb 460. A combination of techniques such as radio frequency (RF) shaping and grinding may then be employed to obtain the final shape of the flow control element 464. This may comprise flow control ramps for both the first orifice 454 and the second orifice 456, as well as the other features described above with respect to other embodiments.

Various other considerations may affect the specific details of the design and construction of the improved catheter tip according to embodiments of the present invention. For example, the tip should not cause a jump in the outer diameter of the catheter, which might preclude using the improved device in certain applications. Accordingly, the maximum radial dimension of the tip is preferably substantially the same or smaller than the radius of the distal portion of the catheter using the tip. Similarly, the tip portion does not restrict catheter passage through an introducer sheath. The tip also is designed to prevent obstructing the passage of a guidewire. A guidewire that may be used with the base catheter is thus also usable with the catheter plus the distal tip. As the embodiments of the distal tip also require no greater than normal pressure to pass fluid therethrough, no changes are required to the supporting equipment. In addition, the improved tip has hemolysis and thrombogenesis characteristics at least as good as those of conventional catheters. Red blood cells are not excessively damaged by traveling through the exemplary tip, and the formation of thrombi is not increased.

Exemplary embodiments of the distal tip according to the invention have been tested and compared to a conventional silicone staggered tip dialysis catheter. An improved tip formed of a single molded element attached to a distal end of a catheter as described above was tested, as well as an improved carbothane tip bolus overmolded on a carbothane catheter. All the catheter and tip combinations had a diameter of 15 Fr, and were compatible with a 0.038″ guidewire. The arterial and venous flow rates for the conventional catheter were both about 155 ml/min. The improved molded tip produced arterial and venous flow rates of about 220 ml/min, while the improved carbothane overmolded tip resulted in arterial and venous flow rates of about 285 ml/min and 295 ml/min, respectively.

Both of the improved tips resulted in much improved reverse recirculation rates when compared to the conventional tip catheter. The conventional catheter had a recirculation rate of about 0.4% in the normal mode and about 20.9% in reverse mode. The molded silicone improved tip showed a normal mode recirculation rate of about 2.4% and a reverse mode rate of about 6.3%. The overmolded carbothane tip had a normal mode recirculation rate of about 0.7% or less, and a reverse mode recirculation rate of about 10% to 14%. The exemplary improved tips resulted in a slightly higher level of PFHB hemoglobin, indicating slightly higher hemolysis, or damage to the blood's cells. The base catheter levels were about 8.04, compared to about 8.11 for the molded silicone improved tip. Both improved tips were less thrombogenic than the conventional catheter.

The present invention has been described with reference to specific embodiments, and more specifically to a dialysis catheter with dual lumens. However, other embodiments may be devised that are applicable to different medical devices, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

1. A distal tip for a catheter comprising:
first and second lumens extending therethrough, wherein in an operative configuration, the first and second lumens are coupled to first and second lumens of a dual lumen catheter;
a first opening fluidly connected to the first lumen for inflow of fluid from a body lumen into which the distal tip is inserted in a normal mode of operation and for outflow of fluid thereto in a reverse mode of operation;
a second opening fluidly connected to the second lumen, the second opening being disposed distally from the first opening and separated therefrom by a selected stagger distance for outflow of fluid therefrom when the catheter is in the normal mode of operation and for inflow of fluid from the body lumen in a reverse mode of operation;
a contoured flow deflection element directing, in the reverse mode of operation, outflow from the first opening away from the second opening; and
a contoured outlet portion of the second opening reducing an outflow velocity therefrom in the normal mode of operation.

2. The distal tip according to claim 1, wherein the first and second openings are disposed on opposite sides of the distal tip with respect to a longitudinal axis thereof.

3. The distal tip according to claim 1, wherein the first and second openings have orifices extending in planes angled with respect to a longitudinal axis of the distal tip.

4. The distal tip according to claim 1, wherein the contoured flow deflector element is adapted to direct outflow from the second opening away from the first opening in the normal mode of operation.

5. The distal tip according to claim 1, further comprising an atraumatic tip formed at a distal end of the distal tip.

6. The distal tip according to claim 1, wherein the first opening includes a first ramp portion deflecting outflow therefrom away from a longitudinal axis of the distal tip in the reverse mode of operation.

7. The distal tip according to claim 6, wherein the first ramp comprises side extensions preventing outflow from spilling radially around the distal tip.

8. The distal tip according to claim 1, wherein the second opening includes a second ramp portion deflecting outflow from the second opening away from a longitudinal axis of the distal tip in the normal mode.

9. The distal tip according to claim 1, wherein the second opening comprises an expanded section increasing an exit plane cross sectional area of the second orifice.

10. The distal tip according to claim 1, wherein the first and second lumens have substantially D shaped cross sections.

11. The distal tip according to claim 1, further comprising a contoured bolus including a first ramp substantially aligned with the first opening, a second ramp aligned with the second opening and an atraumatic distal tip.

12. The distal tip according to claim 11, wherein a maximum radial dimension of the contoured bolus is less than a radius of a catheter to which the distal tip is to be coupled.

13. The distal tip according to claim 1, wherein the selected stagger distance is between about 1.0 cm and 1.5 cm.

14. The distal tip according to claim 11, wherein a maximum radial dimension of the contoured bolus is substantially the same as a maximum radius of the distal tip.

15. The distal tip according to claim 1, wherein the second opening has a dimension substantially equal to a dimension of the first opening.

16. A flow control tip for a multi-lumen catheter comprising:
an attachment portion adapted to fluidly connect to a distal portion of a catheter; and
a contoured bolus defining at least a portion of an inlet and an outlet of the distal tip so that, when coupled to a catheter, the inlet is coupled to a first one of the catheters lumens and the outlet is coupled to a second one of the catheters lumens, and a flow deflector directing fluids exiting the inlet in a first mode away from the outlet, wherein the contoured bolus defines a specified stagger distance between the inlet and the outlet.

17. The flow control tip according to claim 16, wherein the contoured bolus further comprises a second flow deflector directing fluid exiting the outlet in a second mode away from the inlet.

18. The flow control tip according to claim 16, wherein the inlet and the outlet are formed on opposite surfaces of the contoured bolus.

19. The flow control tip according to claim 18, wherein the flow deflector comprises a ramp disposed adjacent an inlet opening.

20. The flow control tip according to claim 18, wherein the contoured bolus defines an expanded section at the outlet increasing an exit plane cross-sectional area of the outlet.

21. The flow control tip according to claim 20, wherein a size of the expanded section is selected to reduce an exit pressure of the fluid to a predetermined level.

22. The flow control tip according to claim 20, further comprising a split in a distal end of the flow control tip cooperating with the expanded section to increase the exit plane cross-sectional area of the outlet.

23. The flow control tip according to claim 16, wherein the attachment portion is adapted for attachment to the catheter by one of a mechanical fitting, a friction fitting, chemical bonding and thermal bonding.

24. The flow control tip according to claim 16, wherein at least portions of the flow control tip are formed integrally with the catheter.
